(19) United States
(12) Patent Application Publication
(10) Pub. No.: US 2003/0211551 A1
(43) Pub. Date: Nov. 13, 2003

(54) Platelet function assay and reagent therefor

(76) Inventors: Donald E Mahan, Raleigh, NC (US); Michael W Stewart, Alberta (CA)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,766

(22) PCT Filed: May 9, 2001
(86) PCT No.: PCT/US01/11760

(51) Int. Cl. G01N 33/567; C12Q 1/56

(52) U.S. Cl. 435/7.21; 435/13

(57) ABSTRACT

A platelet function assay reagent is provided for performing a platelet function assay, wherein the reagent contains a mixture of magnetic and non-magnetic particles, wherein the magnetic particles have bound to an outer surface thereof an amount of a first ligand having an affinity for direct interaction with GP-Ib receptors on blood platelets and wherein the non-magnetic particles have bound to an outer surface thereof an amount of a second ligand having an affinity for direct interaction with GP-Ib receptors on blood platelets, such that interaction of either of the first or second ligands with the GP-Ib platelet receptor will activate the blood platelets toward aggregation, wherein the first ligand and the second ligand can be the same or different, and the assay using such reagent, for providing a fast, reliable point-of-care assessment of platelet function.

### BACKGROUND OF THE INVENTION

1. Field of the Invention

The present invention relates to a platelet function assay allowing for point of care assessment of GP IIb/IIIa receptor dependent platelet aggregation, and the reagents therefor.

2. Discussion of the Background

The physiological response to vascular injury involves the binding of platelets to exposed extracellular matrix (adhesion) followed by platelet recruitment to form a platelet plug (aggregation). Study of the haemostatic process has led to the development of methods, which examine either adhesion or aggregation. Historically, platelet function has been evaluated by stirring platelets in the presence of soluble agonists. Previous studies investigating the role of platelets in maintaining haemostasis have suggested that platelet contact with subendothelium is an early event in establishing platelet adhesion at the site of vascular injury (Sakariassen et al, British J. of Haematology, 63, 681-691, (1986)). Platelet adhesion to immobilized substrate has been investigated under static (Savage et al, J. Biological Chemistry, 267, 113000-11306, (1992)) and flow conditions (Olson et al, J. of Laboratory and Clinical Medicine, 14, 6-18, (1989)); however, these techniques do not address platelet-platelet interaction. No current method addresses both adhesion of platelets to extracellular matrix and subsequent platelet-platelet interaction.

Platelets also participate in a number of fundamental aspects within the coagulation cascade. Thrombin, the principle product of the clotting cascade, is a potent platelet activator and results in the aggregation of platelets at the site of vascular injury. Once enmeshed in the network of a fibrin clot, platelet contraction furnishes the force responsible for clot retraction. Platelets also provide a surface for assembly of coagulation complexes responsible for promoting thrombin generation. Platelet aggregation is mediated through the binding of fibrinogen to glycoprotein receptors IIb/IIIa (GP IIb/IIIa) on the platelet surface and platelet adhesion is mediated through the glycoprotein receptor Ib.

With the introduction of Reopro™ by Centocor in 1993 a new class of therapeutic agent was added to the medical arsenal for haemostasis control. This blocker of the platelet GP IIb/IIIa receptor has been found effective in preventing ischemic complications of unstable angina and percutaneous coronary interventions. The success of Reopro™ has prompted the development of other GP IIb/IIIa blocking agents and, as of 2000, eptifibatide (Cor Therapeutics) and tirofiban (Merck) have received U.S. Food and Drug Administration approval for use. Each of these new therapeutics agents differ in their pharmacodynamic profiles. Data from the EPIC study suggests that to be of benefit there is a need to achieve and sustain for 12 hours 80% blockade of platelet glycoprotein IIb/IIIa receptors. It is not unreasonable to expect that differences between individuals may influence effective therapy with these new compounds. Platelet count, GP IIb/IIIa surface density, and drug clearance by the liver or kidney may influence the effective dose and/or dosing regimes for these new anti-platelet agents. Periodic monitoring may also provide critical information regarding effective therapeutic levels during prolonged operative procedures and recovery.

Instruments on the U.S. market capable of measuring platelet aggregation include such instruments as the Platelet Function Analyzer (PFA-100™, Dade), the Clot Signature Analyzer (CSA®, Xylum) and the Optical Platelet Aggregometer (Chrono-Log Corp.). Although these systems may respond in some manner to GP IIb/IIIa blocking agents, they were designed to assess the global characteristics of platelet-dependent haemostasis and not specifically GP IIb/IIIa receptor function. Furthermore, these methods require expensive equipment, skilled operators in laboratories often available only on weekdays during regular working hours, and extensive maintenance to ensure accuracy and standardization of results. One company, Accumetrics of San Diego, Calif. has developed a light transmittance device for the point-of-care market designed specifically to assess the number of unblocked platelet glycoprotein (GP) IIb/IIIa receptors in a whole blood sample (U.S. Pat. No. 5,552,290). The Ultegra™ instrument measures a change in an optical signal due to fibrinogen-coated particle agglutination in the sample and reports a semi-quantitative result. Each of these systems uses a soluble agonist to activate the platelets leading to an aggregation response in a sample drawn from a patient. A solid phase agonist such as von Willebrand factor immobilized on polystyrene beads can also be used to activate platelets as described by Shaw and Stewart (U.S. Pat. Nos. 5,952,184 and 5,427,913, hereby incorporated by reference).

The Thrombolytic Assessment System (TAS™) analyzer, manufactured by Pharmanetics (Morrisville, N.C. 27560) measures the kinetics of fibrin polymerization following activation of the coagulation pathway in a patient's blood sample. The TAS™ analyzer and disposable were designed for use with whole blood in a point-of-care setting. Paramagnetic iron oxide particles (PIOP) are an essential component of the detection system for each of the tests developed for the TAS™ analyzer. The PIOP and other lyophilized ingredients for a particular test are located in the shallow reaction chamber of the TAS test card disposable. In addition to PIOP, the test reagent may contain buffers, stabilizers, fillers and specific coagulation pathway activator or agents. A test is initiated by insertion of a dry-chemistry test card into a slot of the TAS™ analyzer that automatically positions the test card reaction chamber above an electromagnet. This chamber is also illuminated with infrared light from a light emitting diode. The instrument measures reflected infrared light from the surface of the test card by means of a solid state photodiode detector. A test is automatically initiated when the analyzer photodetector measures a change in reflected light intensity when blood or plasma is added to the sample well of the test card and, through capillary action, is pulled into the reaction chamber. The activators present in the reaction chambers stimulate the coagulation cascade in the patient's sample to produce thrombin, which in turn catalyzes the formation of the fibrin clot.

During a clotting test the TAS™ analyzer electromagnet oscillates on and off every second. The magnetic particles stand up when the electromagnet is on, causing more light to be reflected to the detector, and fall down when it is off, causing less light to be detected. This movement of PIOP produces an alternating current (AC) signal from the photodetector. As the test proceeds, more and more fibrin polymerization occurs and the PIOP movement is less. The analyzer in accordance with predetermined algorithms interprets the signal produced by the relative movement of the PIOP and reports an endpoint (clotting time) appropriate for each test.

Although PIOP is an integral component of the TAS™ detection system, it does not, by design, participate directly in activation of the coagulation cascade or fibrin polymerization. To prevent undesired interactions between PIOP and activators within the reaction chamber of a test card, the PIOP is coated or blocked with bovine serum albumin (BSA). BSA is a protein commonly used by those skilled in assay development to prevent unwanted interactions between surface components of a test and its active ingredients. The TAS™ system was designed to monitor fibrin polymerization and not platelet aggregation.

### SUMMARY OF THE INVENTION

Accordingly, one object of the present invention is to provide an assay for platelet function that can be used on whole blood with ease of detection in a single stage method.

A further object of the present invention is to provide an assay for platelet function that can be used on the TAS™ system for rapid determination of platelet function.

A further object of the present invention is to provide a platelet function assay reagent, preferably in a dry chemistry test card format that can be used in the platelet function assay of the present invention.

A further object of the present invention is to provide a platelet function assay that assesses both platelet adhesion to an extracellular matrix and subsequent platelet-platelet aggregation.

These and other objects of the present invention have been satisfied by the discovery of a platelet function assay reagent comprising magnetic particles and non-magnetic particles, wherein both the magnetic particles and non-magnetic particles have bound thereto an amount of a ligand that directly interacts with GP-Ib receptors on blood platelets sufficient to activate the platelets toward GP-Ib/IIIa receptor dependent platelet aggregation, and its use in a platelet function assay wherein the reagent is arranged in a substantially flattened configuration and said magnetic particles and non-magnetic particles are distributed substantially homogeneously therethrough, such that upon addition of a whole blood or platelet rich plasma sample, both types of particles are freed with the magnetic particles moving in response to an applied oscillating or rotating magnetic field, with the assay endpoint determined by monitoring the motion of the magnetic particles to determine platelet function.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein;

FIG. 1a shows the PIOP ring formed during the assay of the present invention using a rotating magnetic field.

FIG. 1b shows the PIOP ring formed during the assay as it begins to collapse toward the center point of the ring.

FIG. 1c shows the disc or dot formed from complete collapse of the PIOP ring in the presence of a rotating magnetic field.

FIG. 2 is a three dimensional plot of the magnetic field strength of the stationary magnetic assembly of a preferred embodiment of the present invention.

FIG. 3 is a graph of DC voltage measurements from the TAS recorded over time during whole blood platelet function tests of an embodiment of the present invention.

FIG. 4 is a graph showing the correlation of DC voltage measurements recorded at the conclusion of the preferred embodiment of platelet function test of the present invention on the TAS analyzer and electrical impedance measurements made with the Chrono-Log instrument.

### DETAILED DESCRIPTION OF THE PREFERRED EMBODIMENTS

The present invention relates to a platelet function assay reagent and its use in a platelet function assay. The platelet function assay of the present invention monitors GP IIb/IIIa receptor dependent platelet aggregation. The assay can be performed either in a wet chemistry format or a dry chemistry format.

The assay reagent of the present invention comprises two central reagent elements. The first is magnetic particles, preferably paramagnetic iron oxide particles (PIOP), such as those currently employed in the TAS™ analyzer (described in U.S. Pat. Nos. 4,849,340; 5,110,727; 5,350,676; 5,601,991; 5,670,329; and 5,677,233, each of which is hereby incorporated by reference), which have been modified by binding a ligand to their surface that can interact directly with the GP-Ib receptor on blood platelets. While a variety of magnetic particles can be used, as described in U.S. Pat. No. 5,110,727, the preferred magnetic particles are PIOP. Accordingly, the following description will refer to PIOP for convenience, but it is to be understood that unless otherwise indicated, the term PIOP can refer to any magnetic particles type. The PIOP, as in the conventional TAS applications, also plays a central role in the assay monitoring and detection system, whereby movement of the modified PIOP in response to a moving magnetic field is monitored to determine the endpoint of platelet aggregation.

The second central reagent element is non-magnetic beads or microspheres also coated with a ligand that can interact directly with the GP-Ib receptor on blood platelets. These non-magnetic beads or microspheres can be any desired particle size, preferably on the same order of size as the PIOP, more preferably having a particle size of from 1 to 20 microns. The non-magnetic beads can be made from any non-magnetic material that is capable of having a ligand bound to its surface. Preferred materials for preparation of the non-magnetic beads include, but are not limited to, polystyrene beads, polyolefin beads, glass beads, and even non-magnetic metal beads. Most preferred are the polystyrene beads as described by Stewart et al, British J. Haematology, 97, 321-329 (1997) and Shaw et al, U.S. Pat. No. 5,952,184, each of which are hereby incorporated by reference. The purpose of the non-magnetic bead is to provide a second site for platelet interaction. Other reagents known to those skilled in the art to enhance functional stability, reagent drying and material rehydration on sample addition may also be added to the test formulation, such as anticoagulants, buffers, etc, and as described in the Oberhardt, and Shaw patents described herein.

The ligand can be bound to the particles directly or indirectly through a spacer, so long as the activity of the ligand is not impaired. The direct binding can occur either covalently or non-covalently. Indirect binding can occur through spacers, including but not limited to peptide spacers, antibody spacers or carbohydrate spacers. These spacers normally act only as bridges between the particle and the ligand, but could be used in order to alter the effectiveness of the ligand/receptor interaction. For example, coupling vWf to the particle through a 7 amino acid peptide bridge could decrease the interaction of vWf with the receptor. However, use of the active segment of vWf, coupled to the particle through the same 7 amino acid peptide bridge could result in upregulation of the vWf fragment/receptor interaction. Similar enhancements of other types have been seen by Beer et al, Blood, 79, 117-128 (1992).

In one embodiment of the present invention assay, the reagent can contain only the magnetic particles when the oscillating magnetic field is used. However, as described below, when the rotating field is used, the reagent must contain both magnetic and non-magnetic particles having ligands bound to both types of particles in order to obtain a meaningful result. The reagent composition contains the above noted magnetic and non-magnetic particles coated with the ligand, preferably vWf, in amounts such that the reagent composition comprises 1-2 mg of coated PIOP particles per ml and from 2×10⁶ to 8×10⁶ polystyrene particles per ml. Once the reaction chamber is filled, the sample is then frozen and lyophilized, as described for the preparation of test cards in the above noted Oberhardt patents.

The ligand that interacts directly with the GP-Ib receptor on blood platelets can be any compound capable of performing that function, that results in activation of the platelets toward GP-IIb/IIIa receptor dependent platelet aggregation. Suitable ligands include, but are not limited to, von Willebrand factor (vWf), collagen and thrombin, as well as fragments (also known as mimetopes, such as those described by Miller, U.S. Pat. No. 5,877,155, hereby incorporated by reference) thereof. Most preferred is the use of von Willebrand factor or a fragment thereof as the ligand.

The ligand used on the non-magnetic particles and the magnetic particles can be the same ligand or different ligands. Preferably, the ligand is the same on both types of particles, most preferably von Willebrand factor or a fragment thereof. The ligand should be present on the surface of the magnetic and non-magnetic particles in an amount sufficient to provide binding to the GP-Ib receptor, and activation of the platelets, such that a sufficient number of platelets are activated to result in an assay endpoint within a time period of from 1 to 20 minutes, preferably from 2-4 minutes. For example, in the case of the rotating magnetic field as described below, the endpoint is reached when the initially formed rotating PIOP ring collapses into a solid disc or dot.

The assay of the present invention can be either in a wet chemistry or dry chemistry format. In either format, the test can be performed on a relatively flat reaction surface, preferably in a reaction slide such as that described in the above-noted Oberhardt U.S. Patents. Most preferably, the assay is performed in a dry chemistry format using a reaction slide or card as described in U.S. Pat. No. 5,110,727, the contents of which are hereby incorporated by reference. The present assay can also be adapted for use in disposables with reagent chambers of any desired size and shape.

To perform the platelet function assay of the present invention, it is necessary to place the reaction chamber into an oscillating magnetic field, such as that described by U.S. Pat. No. 5,110,727 (previously incorporated by reference) or into a rotating magnetic field, such as that described in U.S. Pat. No. 5,670,329 (hereby incorporated by reference). Most preferably, the assay is performed in the presence of the rotating magnetic field. The magnet (either oscillating field type or rotating field type) must be designed such that the field can influence substantially all of the PIOP present in the reaction chamber. In the preferred example of the rotating magnetic field, when the reaction chamber is that present in a TAS test card, the separation between the magnetic poles may range from 0.5 to 2.5 cm. The magnet must be positioned sufficiently close to the reaction chamber to cause movement of PIOP when the magnetic field is rotating. The rotating magnetic field can rotate at any frequency capable of sustaining the circular movement of the magnetic particles of the system, with the rotational frequency being preferably from 2,000 to 3,000 rpm. The rotating magnetic field can be provided by the rotation of a permanent magnet about a central axis as described in U.S. Pat. No. 5,670,329, or can be generated by sequential activation of a series of electromagnets in a circular arrangement, as also described in U.S. Pat. No. 5,670,329, the contents of which are hereby incorporated by reference.

One design for the magnet for providing a rotating magnetic field in the present assay comprises two sets of button magnets mounted on a metal disk approximately 3.4 cm in diameter. The metal disk base is attached at its center to the shaft of a DC electric motor. Each button magnet assembly contains three readily available button magnets of approximately 1 cm in diameter. The button magnets are positioned directly opposite of one another on the metal base. On the top of each button magnet assembly is placed one-half of a second metal disk (semi-circle, half of a circle) approximately 3 mm thick with a radius of 9 mm. The straight edges of the two disks face one another and are separated by approximately 1.5 cm. The entire assembly is positioned approximately 2-4 mm beneath the reaction chamber of a test card.

The assay is initiated by addition of a whole blood or platelet rich plasma sample to a reaction chamber containing the reagents described, positioned above the magnet, preferably generating a rotating magnetic field most preferably at a rotational frequency of 2500 rpm. In the most preferred embodiment using a dry chemistry format, the reagent is re-hydrated by the sample, freeing the magnetic particles and allowing them to begin to move in response to the rotating magnetic field. In the presence of the rotating field, the magnetic particles organize as a ring or band of dark material traveling along the outer edges of the reaction area. The center of the ring initially is clear or slightly gray (i.e. contains substantially fewer PIOP). The non-magnetic particles are preferably chosen so as to be invisible to the detection system.

In a sample that does not contain a GP IIb/IIIa inhibitor the band of PIOP becomes smaller over a period of a few minutes and the center of the ring fills in with PIOP to form a solid dot in the center of the reaction area. Microscopic examination of the PIOP from the solid dot reveals that it is an aggregate of PIOP and non-magnetic beads with none or few free platelets or beads in the field of view. If the sample contains a GP IIb/IIIa inhibitor, such as Reopro©, the initial PIOP ring does not collapse to form a solid dot even after a prolonged incubation. Microscopic examination of the PIOP in such a sample shows few or no non-magnetic beads associated with PIOP. If the non-magnetic beads are left out of the test card formulation, collapse of the PIOP ring does not occur. The non-magnetic beads themselves are preferably not readily visible in the system and do not participate in determining an end point for the assay. The end point of the test is established by the position and mobility of the PIOP present in the reagent formulation. Motion of the PIOP in the reaction area imparted by the rotating magnetic field is required to activate the platelets through contact with the solid phase agonist (i.e. the ligand) present on both the PIOP and non-magnetic beads. The aggregation of the two solid phases occurs through platelet adhesion to the solid phase and then platelet activation leading to platelet/platelet binding (platelet aggregation). Aggregation does not occur in the absence of platelets or in the presence of a GP IIb/IIIa inhibitor.

The present invention platelet function assay using a ligand such as von Willebrand factor, coated on a solid phase support (solid-phase agonist) is unique since it addresses both adhesion of platelets to extracellular matrix and subsequent platelet-platelet interaction.

Interestingly, if an antibody to the GP-Ib receptor is used to block the GP-Ib receptor from interaction with the ligand on the particles in the system, no reaction between the platelets and modified particles occurs and no platelet/platelet aggregation occurs. It is only upon activation of the platelets by the direct interaction of the ligand with the GP-Ib receptor that one can obtain platelet/platelet aggregation and provide a meaningful assay. While it has been noted in the past that von Willebrand factor has the ability to bind either the GP-Ib or the GP-IIb/IIIa receptors, the binding to the GP-IIb/IIIa receptor can only occur if the platelet has been activated. Without that activation, there is little or no interaction between vWf and the GP-IIb/IIIa receptor. This is significantly different from the Coller work (such as described in U.S. Pat. Nos. 5,763,199 and 5,854,005) which inherently requires that the platelets be activated such that the receptor ligand bound to the solid surface interacts with the GP-IIb/IIIa receptor to initiate the assay.

In the assay of the present invention, once the bound ligand has interacted with the GP-Ib receptor and activated the platelets, the free fibrinogen naturally in the sample interacts with the GP-IIb/IIIa receptors on the platelets, causing platelet/platelet aggregation. As the platelets aggregate, this increases the effective mass of the PIOP/microsphere aggregates, causing the heavier aggregates to migrate inward from the outer ring of particles toward the center of the rotating magnetic field. As the assay progresses, the ring eventually collapses into a circular dot, which continues to rotate about the center of the rotating field.

The end point of the assay can be monitored by using reflected infra red light similar to that of the TAS analyzer. This is possible because the area of the reaction chamber covered by the dark ring is much greater than that of the solid dot. The signal can provide both a qualitative YES/NO response and a quantitative response. The qualitative response provides an indication of whether a blood sample has the GP-IIb/IIIa receptors blocked to an extent sufficient to prevent aggregation (which results in no ring collapse) or whether the sample is within normal limits such that the sample donor has sufficient ability to form thrombi and undergo platelet aggregation. The signal can also be analyzed and compared to a standard curve to determine the percentage of GP-IIb/IIIa receptor blockage, thereby providing a more accurate ability to determine dosage levels of medications required for the patient.

While the present invention assay is particularly useful for platelet function tests for patients having normal or reduced platelet function, it can also be used in cases where the patient has hyper-platelet function. This condition particularly occurs in patients receiving anti-IIb/IIIa agents, such as stroke patients, unstable angina patients, and acute myocardial infarction patients. Hyper-platelet function translates to either an activated state of the platelet (i.e. GP IIb/IIIa is already activated and can bind fibrinogen and/or vWf) or the propensity of the platelet to activate under circumstances in which it would normally remain quiescent.

In the former case (platelets are pre-activated) the platelets can bind directly to the bound ligand of the present invention, such as vWf, through the GP IIb/IIIa receptor. However, this interaction is not sufficient to cause collapse of the PIOP ring into the dot or disc. This has been demonstrated by coating the particles of the present invention with fibrinogen (active towards the GP IIb/IIIa receptor, not the GP Ib receptor). Upon using the fibrinogen coated particles, the PIOP ring formed in the presence of the rotating magnetic field does not undergo the complete collapse to the central point of the ring, but rather only undergoes a partial contraction of the ring. Accordingly, further activation of the IIb/IIIa receptors are required for a successful assay. Viewed another way, if the IIb/IIIa receptors were already activated sufficiently in the sample, the platelets would have already undergone aggregation and been removed by the reticuloendothelial system.

Accordingly, the present invention can be used not only to activate platelets by interaction at the Ib receptor, but also to potentiate the activation of hyper platelet function by further interaction at the Ib receptor. This preactivation in a hyper platelet function sample can be due to other agonists, such as collagen, arachidonic acid, adrenaline, ristocetin, thrombin, or TRAP, particularly at sub-threshold concentrations (where no platelet aggregation is found). The combination of the subthreshold concentration of the agonist and the interaction of the bound ligand of the present invention at the Ib receptor provides for sufficient platelet activation to cause aggregation and consequently, the collapse of the PIOP ring into the disc or dot.

The difference in signal produced by a ring versus a dot can be enhanced if a small spot of reflective material covers the very center of the reaction area. In this situation the dark ring of PIOP will disappear behind the reflecting material, thereby increasing the signal. Alternately the end point of the assay could be monitored by a video or infra red camera. The output of the camera can be digitized and the image subsequently analyzed to determine the formation of ring and dot structures.

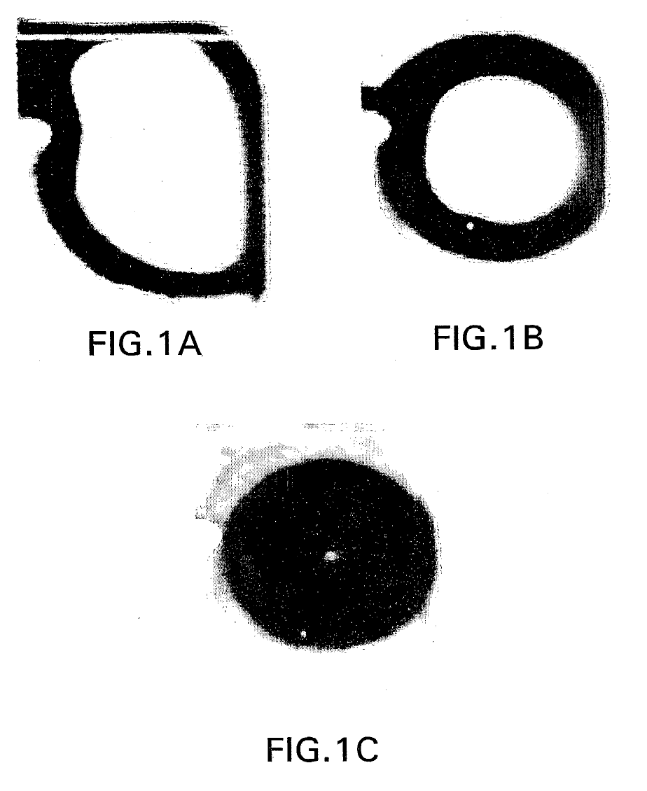

FIG. 1a provides a representation of the assay of the present invention in operation using a rotating magnetic field with an assay test card such as that from U.S. Pat. No. 5,110,727. In this Figure, the PIOP have been freed by the addition of the blood sample, resulting in the formation of a rotating ring (10) of PIOP within the reaction chamber (20).

FIG. 1b shows the rotating ring (10) of PIOP has started to collapse toward the center point (30) representing the axis of the rotating field. FIG. 1c shows the endpoint of the assay in which the PIOP have completely collapsed into a dot structure (40). This endpoint is dramatic and readily detectable both instrumentally and visually. Visual detection provides a quick and easy method for obtaining the qualitative information discussed above, while instrumental detection using a system such as the TAS analyzer, provides the ability to analyze the signal obtained and provide quantitative measures of receptor blockage, preferably by comparison to a standard curve generated using samples of known receptor blockage percentage.

When an oscillating magnetic field is used, the signal produced is analyzed in the same manner as in Oberhardt U.S. Pat. Nos. 5,110,727 and 4,849,340, by analysis of the decay curve produced by monitoring the oscillation of the particles. Additionally, when using the oscillating magnetic field, is it possible to use only magnetic particles with the bound GP-Ib receptor ligand on their surface, without the need for the modified non-magnetic particles.

Having generally described this invention, a further understanding can be obtained by reference to certain specific examples which are provided herein for purposes of illustration only, and are not intended to be limiting unless otherwise specified.

### EXAMPLES

Sample Preparation. Whole blood (4.5 mL) was drawn by venipuncture into Vacutainer tubes (Becton Dickinson No. 366415, Lot 0018242) containing 0.5 mL of 3.2% buffered citrate (0.105M). Platelet poor plasma (PPP) was prepared by centrifugation of whole blood at 2,000 g for 15 minutes and carefully removing the straw colored supernate from the packed red blood cell pellet with a disposable pipette. PRP was prepared by centrifuging whole blood at 150 g for 10 minutes.

Impedance Aggregation. Whole blood impedance aggregation was performed using a model 560-CA dual channel Chrono-Log Lumi-Aggregometer as described by the manufacturer, see also Mascelli, et al⁹. An aliquot of whole blood (0.5 mL) was diluted with an equivalent volume of sterile saline and incubated for 5 minutes at 37° C. in a cuvette (Chrono-Log Cat#367) containing a small stir bar (Chrono-Log Cat#370). Electrodes were inserted into the sample and the baseline impedance of the sample adjusted to zero. Aggregation was initiated by addition of 5 uL of 1 mg/mL collagen (Chrono-Log, Cat#385) and platelet aggregation monitored over 5-6 minutes. Impedance output was captured via an Aggro/Link® interface and the results stored on the computer's hard drive. For ex vivo samples treated with ReoPro, the percent aggregation was determined by dividing the maximum change in ohms of the test sample by the maximum change in ohms of a baseline sample (zero ReoPro) and multiplying the result by 100.

Preparation of vWf Antihemophilic Factor (Human), Alphanate™, was obtained from the Alpha Therapeutic Corporation, Los Angeles, Calif. Alphanate™ is prepared by cryoprecipitation and affinity chromatography of the Factor VIII from pooled human plasma. vWf is co-purified along with Factor VIII as part of this purification process. The product of the purification process is heated and chemically treated to reduce the potential risks of viral transmission. Alphanate™ is intended for the prevention and control of bleeding in patients with Factor VIII deficiency due to hemophilia or acquired Factor VIII deficiency. Alphanate™ is supplied in sterile, lyophilized form in a single dose vial (˜1000 IU/vial) along with a separate 10 mL vial of sterile water for injection. A unit of vWf is defined as that amount found in 1 mL of pooled normal plasma. The preferred storage temperature for Alphanate™ is 4° C.; however, the product may be kept at room temperature (not to exceed 30° C.) for up to 2 months.

Alphanate™ was reconstituted by first allowing the vials of Alphanate™ and sterile water to warm to room temperature. Ten milliliters of sterile water was gently added to the lyophilized vial of Alphanate™ concentrate and the container allowed to stand until its contents were dissolved. It was necessary to gently swirl the liquid in the vial until all the lyophilized cake was in suspension. Reconstitution required less than 5 minutes. The rehydrated Alphanate™ was dialyzed overnight against phosphate buffered saline (0.01M phosphate, pH 7.2, 145 mM NaCl₂) using Spectra/Por Membrane (Cat#132 128) with a molecular weight cutoff of 50,000. The dialyzed material, approximately 10 mL of ˜10 IU/mL, was divided into 1 mL aliquots and stored frozen at −80° C. if not used immediately. Each mL of dialyzed protein was sufficient to prepare ˜2.3 mL of vWf-coated paramagnetic iron oxide particles (PIOP) or ˜1.6 mL of vWf-coated polystyrene beads. A single vial of Alphanate™, therefore, was sufficient to manufacture 6,000 test cards.

Preparation of vWf-coated PIOP. 10 g of Magnetite obtained from ISK Magnetics (Valparaiso, Ind.) was added to 90 mL of 50 mM Tris pH 7.4 in a sealed flask and purged with nitrogen for 5 minutes. The PIOP suspension was then homogenized by a model 700 PowerGen Homogenizer (Fisher Scientific) for 5 minutes at a setting of 6 using a 7 mm diameter rotor-Stator. A 0.7 mL aliquot of the homogenized PIOP was added to 0.3 mL of approximately 10 ug/mL vWf and allowed to incubate for 30 minutes at room temperature.

Preparation of vWf-coated polystyrene beads. Polystyrene beads (4 um) obtained from Polysciences Corporation (Warrington, Pa.) were washed three times with 0.2 mol/l carbonate buffer (pH 9.35) prior to use. vWf was diluted in 0.2 mol/l carbonate buffer (pH 9.35) to 2 U/mL (a unit of vWf is defined as the amount found in 1 mL of pooled normal plasma) and mixed with polystyrene beads pre-equilibrated in carbonate buffer prior to incubation overnight at 4° C.

Preparation BSA-coated PIOP. 10 g of Magnetite obtained from ISK Magnetics (Valparaiso, Ind.) was added to 90 mL of 50 mM Tris pH 7.4 in a sealed flask and purged with nitrogen for 5 minutes. The PIOP suspension was homogenized using a model 700 PowerGen Homogenizer (Fisher Scientific) for 5 minutes at a power setting of 6, using a 7 mm diameter Rotor-Stator (Cat#15-338-205). The power setting of the homogenizer was reduced to 3 and 8 mL of 100 mg/mL BSA added dropwise in 10-400 uL aliquots. The power setting was reduced to 2 and mixed for 30 minutes. The particle suspension was poured into a 100 mL graduated cylinder and the particles allowed to settle for 20 minutes. The supernate was removed (˜70mL), replaced with 50 mL Tris pH 7.4 and mixed by inversion. The wash step was repeated four times, and after the final wash, the particles were resuspended in 80 mL of 50 mM Tris pH 7.4.

Formulation of Platelet Function Test Card Reagent. Platelet function test card reagents were prepared that varied in the amount of vWf-coated polystyrene beads and content of vWf-coated PIOP. In the following sections, the actual amounts of vWf-coated polystyrene beads and PIOP used in the experiment are described. Cards with desirable properties were prepared when each milliliter of the test card formulation contained 0.033 mL of vWf-coated polystyrene beads and 0.033 to 0.1 mL of vWf-coated PIOP. The remainder of the test card formulation was made up of buffers, stabilizers and bulking agents. The final concentration of these agents in the test formulations was: 30 mM Hepes pH 7.8, 100 mM Trehalose, and 0.2 mg/mL Bovine Serum Albumin (Sigma A-3059). These test card formulations were intended for use with citrated whole blood or plasma. There was no calcium or soluble platelet agonist in the formulation. If a whole blood sample type were desired (i.e. fingerstick, non-citrated sample type), an anticoagulant such as sodium citrate would need to be incorporated into the test card formulation to inhibit fibrin clot formation.

Dry Test Card Preparation—To ensure even filling, standard plasma treated (Gasonics SN 10213) TAS test cards, such as those in Oberhardt, U.S. Pat. No. 5,110,727, were used for the feasibility studies evaluating the platelet function test. The platelet function test card reagent was prepared in 50 mL polypropylene centrifuge tubes (Sarstedt 62.547.004) on the day of filling and the solid components of the formulation kept in suspension by gently swirling the tube by hand. Approximately 30 uL of reagent was manually pipetted into the sample well of test cards to be filled. Once the reaction chamber was filled with reagent, the card was immediately placed into a tray inside of a pre-cooled (˜−190° C.) cryochamber. Trays containing frozen test cards were placed into a lyophilizer and dried. At the completion of the lyophilization cycle, the test cards were stored with a silica gel desiccant at 4° C. until use. During the course of the feasibility study, 36 lots of 20-100 test cards were assembled.

However, the ratio of coated magnetic particles to coated non-magnetic particles is not limited and can be any ratio so long as there are sufficient magnetic particles to form the rotating ring and collapse to the disc or dot.

Hardware. A breadboard instrument, as well as a prototype TAS device, was employed to evaluate the platelet function test. The breadboard device consisted of a fiber optic light source, CDC camera, Video Monitor, VCR tape player and recorder, DC power supply for the motor of the rotating magnet, modified TAS to supply power to the heater unit of the test card holder and a stand for positioning of the test card holder and rotating magnet. The purpose of the TAS analyzer in this setup was to provide power and temperature sensor connections to the modified sensor head assembly holding the test card. The proximity of the rotary magnet assembly to the sensor head assembly and test card was adjusted using a lab jack.

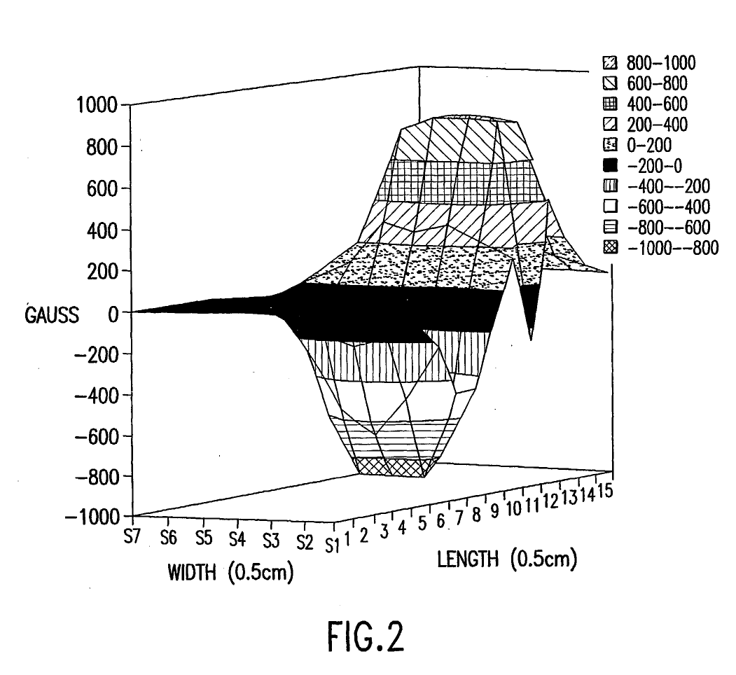

A prototype TAS instrument was assembled by replacing the pulsating electromagnet of the TAS sensor module with a rotating magnet assembly. The rotating magnet was within a plastic housing and mounted to the bottom of a TAS sensor head assembly. The configuration of the magnets in the prototype device was similar to that of the breadboard. Static measurements of the magnetic field strength were made using a FW Bell model 9500 Gaussmeter. These measurements were made by positioning the probe at 0.5 cm intervals along the surface of the test cardholder, starting from the right front corner of the assembly. A three dimensional plot of the field strength measurements is shown in FIG. 2. Each division along the abscissa and ordinate in FIG. 2 represents 0.5 cm. The maximum and minimum Gauss measurements were approximately ±800 units. The peak and valley of the field strength was approximately 1.0-1.5 cm wide and separated by 2.25 cm. A hole was created in the bottom of the case of the TAS analyzer to accommodate the motor and magnetic assembly. An external power supply was used to drive a DC motor at 2,000 rpm (6.5V). The TAS unit provided power to the heating strip of the sensor assembly. Two versions of the prototype were used during the feasibility study that differed in the manner in which the reactions taking place within the test card were monitored.

The first device employed the same sensor head circuit board as that used in the TAS analyzer. This version permitted monitoring of the DC voltage from the sensor head circuit board's solid state detector, which was proportional to the light reflected from the surface of the test card. Because the surface area of the reaction chamber covered by PIOP may change during a platelet function test, the DC voltage output of the detector was one means of monitoring test progress. The DC voltage and time measurements were either recorded by hand over time or later in the program, captured by TAS software and saved to a spreadsheet for computer analysis.

In the second TAS prototype, the upper portion of the sensor assembly was replaced with a specially designed aluminum block. The space immediately above the reaction area of the aluminum block was open to allow image captures of the platelet assay with a CDC camera. A 45° slot was also drilled into the block to accept a fiber optic light source.

Platelet Function Test

The platelet function test was performed by sliding the test card into the slot of the breadboard of the prototype sensor head directly above a rotating magnet turning at 2,000 to 2,500 rpm. The assay was initiated by the addition of a whole blood or platelet rich plasma sample to a test card disposable containing the reagents described above. As the reagent was re-hydrated by the sample, the magnetic particles began to move and organized into a ring or band of dark material traveling along the outer edges of the reaction area (FIG. 1a). The center of the reaction area contained substantially fewer PIOP, and therefore, appeared clear or slightly gray. In a sample that did not contain a GP IIb/IIIa inhibitor, the band of PIOP typically become smaller over a period of a few minutes (FIG. 1b), and the center of the ring filled in with PIOP to form a solid dot in the center of the reaction area (FIG. 1c). Microscopic examination of the PIOP from the solid dot revealed that it was an aggregate of PIOP and polystyrene beads with none or few free platelets or beads in the fields of view. As the platelets aggregated during the test, the effective mass of the PIOP-platelet-polystyrene aggregate increased, causing the heavier aggregates to migrate inward from the outer ring of particles toward the center of the rotating magnetic field. As the assay progressed, the ring eventually collapsed into a circular dot, which continued to orbit around the center of the rotating magnetic field.

Motion of the PIOP in the reaction area imparted by the rotating magnetic field was required to activate the platelets through contact with the solid phase agonist (vWf) present on both the PIOP and non-magnetic beads. The aggregation of the two solid phases apparently occurred through platelet adhesion to the solid phase and then platelet activation leading to platelet/platelet binding (platelet aggregation). This process required that both PIOP and polystyrene solid phases be coated with vWf. Aggregation did not occur in the absence of platelets or in the presence of a GP IIb/IIIa antagonist. If the polystyrene beads were left out of the test card formulation, collapse of the PIOP ring did not occur. The polystyrene beads themselves were not macroscopically visible in the system and did not participate directly in determining the end point for the assay. The position and mobility of the PIOP present in the reagent formulation was responsible for establishing the end point of the test.

If the sample contained a GP IIb/IIIa inhibitor such as ReoPro®, the initial PIOP ring did not collapse to form a solid dot even after a prolonged incubation (10-15 minutes). Light microscopy examination showed few or no polystyrene beads associated with PIOP and many small clusters of polystyrene particles scattered throughout the sample.

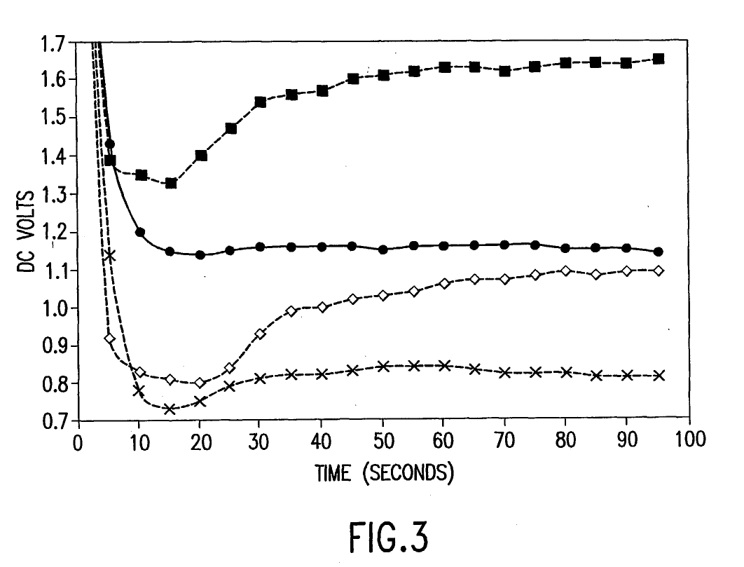

The end point of the assay can be monitored by using reflected infra red light similar to that of the TAS™ analyzer. This was possible because the area of the reaction chamber covered by the dark ring is greater than that of the solid dot. The difference in signal produced by a ring versus a dot can be enhanced if a small spot of reflective material covers the very center of the reaction area. In this situation, the dark ring of PIOP will disappear behind the reflecting material, thereby increasing the signal. This is illustrated in FIG. 3 where DC voltage measurements from the TAS were recorded over time during whole blood platelet function tests using standard TAS test cards (⋄ & ×) and the same cards with a small ⅛″ inch square of white paper in the center of the reaction area (▪ & ●). Two of the test cards (▪ & ⋄) received 30 uL aliquots of whole blood and the remaining two (● & ×) 30 uL of whole blood treated with 10 ug/mL of ReoPro. In each of the tests on sample addition, there was an initial decrease in voltage as the whole blood entered the reaction chamber and the test card reagents were re-hydrated. In the untreated samples, the DC voltage began to increase after 15-20 seconds and reached a plateau after 60-70 seconds. This change in DC voltage was indicative of the PIOP in the test card changing from an open ring structure (FIG. 1a) to one of a solid dot (FIG. 1c). In the ReoPro treated samples, the DC voltage increased only slightly or not at all 15 seconds after sample addition. At 70 seconds, the difference in DC voltage between the untreated and treated samples was 0.21 volts greater for the test cards with the white spot in the center of the reaction area (0.46 vs 0.25). The DC voltage output of the TAS detector is an overall indicator of the light reflected from the surface of the card. The more light reflected from the surface of the test card, the greater the DC voltage.

Alternately, the end point of the assay could be monitored by a video or infra red camera. The output of the camera could be digitized, and the image subsequently analyzed to determine the formation of ring and dot structures. Various spatial aspects of the PIOP structure during a test could be analyzed along with time to provide further information concerning GP-IIb/IIIa receptor blockage or dosage levels for platelet antagonist.

Important elements of the formulation were the number of vWf-coated polystyrene particles in the formulation and amount of vWf-coated PIOP used. This work is summarized in Table 1. Columns two and three of Table 1 indicate the milliliters of vWf-coated PIOP or vWf-coated polystyrene particles used per milliliter of reagent used to fill the test card.

TABLE 1

| Card | mL PIOP/ | mL PS/ | |
|---|---|---|---|
| Lot | mL Rgnt | mL Rgnt | Comments |
| 107 | 0.08 | — | vWf coated PIOP @ RT |
| 108 | 0.10 | — | vWf coated PIOP @ RT |
| 109 | 0.10 | — | vWf coated PIOP @ RT |

Findings: Weak AC signal differences on TAS ± ReoPro. Presence of
calcium in formulation made interpretation of data difficult.

| | | | |
|---|---|---|---|
| 110 | 0.12 | — | |
| 111 | 0.06 | — | BSA coated PIOP in formulation |
| 112 | 0.04 | — | BSA coated PIOP in formulation |
| 113 | 0.06 | — | Plus Fibrinogen coated PIOP |
| 114 | — | — | BSA coated PIOP in formulation |
| 115 | — | 0.06 | BSA coated PIOP in formulation |

Findings: Small differences in AC signal on TAS ± ReoPro using vWF
and fibrinogen coated PIOP. Signal not amplified by use of collagen or
ADP agonist.

| | | | |
|---|---|---|---|
| 116 | 0.008 | 0.067 | |
| 117 | 0.017 | 0.067 | |
| 118 | 0.025 | 0.067 | |
| 119 | 0.008 | 0.10 | |
| 120 | 0.017 | 0.10 | |
| 121 | 0.025 | 0.10 | |
| 122 | — | — | +BSA coated PIOP, Control |
| 123 | 0.025 | — | Control |

Findings: No significant differences noted between formulations with
different levels of vWf-coated polystyrene. Increasing amount of PIOP
increased density of initial PIOP ring and size of final dot.
VWf PIOP and PS required to produce collapse in neat sample.

| | | | |
|---|---|---|---|
| 124 | 0.025 | 0.067 | |
| 125 | 0.067 | 0.067 | |
| 126 | 0.025 | 0.10 | |
| 127 | 0.067 | 0.10 | |
| 128 | — | 0.067 | BSA coated PIOP control |
| 129 | 0.13 | — | Control |
| 130 | — | 0.067 | Fibrinogen coated PIOP control |
| 131 | 0.067 | 0.067 | |

Findings: No collapse observed without polystyrene beads in formulation.
No collapse observed when fibrinogen coated PIOP substituted for vWf-
coated PIOP. Increasing PIOP enlarges size of collapsed dot and increases
density of particles in center of ReoPro treated samples. Performance not
noticeably impacted by different levels polystyrene particles.

| | | |
|---|---|---|
| 132 | 0.083 | 0.10 |

Findings: Wet testing suggested improved perfonnance with higher
amounts of vWf-coated PIOP. Multiple donors tested, collapse not
observed in all untreated samples.

| | | | |
|---|---|---|---|
| 133 | 0.033 | 0.033 | |
| 134 | 0.067 | 0.033 | |
| 135 | 0.100 | 0.033 | |
| 136 | 0.033 | 0.067 | |
| 137 | 0.067 | 0.067 | |
| 138 | 0.100 | 0.067 | |
| 139 | 0.033 | 0.100 | |
| 140 | 0.067 | 0.100 | |
| 141 | — | 0.100 | Fibrinogen coated PIOP added |
| 142 | 0.100 | 0.100 | Collagen 5 ug/mL final |

Findings: Increasing vWf coated polystyrene particles lengthened collapse
time due to consumption of platelets in the assay. Most preferred amount
of vWf coated polystyrene particles was 0.033 mL per mL of reagent.
Increasing concentration of vWf-coated PLOP increased density of initial
PIOP ring and size of collapsed spot. The addition of collagen (5 ug/mL)
to the formulation prevented collapse.

Donor Study

Whole blood samples were drawn from 20 donors, 10 males and 10 females, age 25-53. Any medications taken by the volunteers were recorded. A portion of the untreated sample was used to assess baseline whole blood impedance aggregation on the Chrono-Log analyzer and aggregation on the TAS prototype using dry chemistry test card Lot 134. ReoPro (2 mg/mL stock) was added to aliquots of the ex vivo whole blood sample such that the final concentration to the platelet antagonist was 0.5, 1.0,2.0 or 10 ug/mL. The ReoPro treated samples were incubated for 15 minutes at room temperature prior to testing on the two platelet aggregation instruments. To assess the affect of heparin, unfractionated heparin was added to the whole blood from two donors, numbers 18 and 19.

Each milliliter of reagent used to prepare dry chemistry card Lot 134 contained 0.033 mL of vWf-coated polystyrene beads and 0.067 mL of vWf-coated PIOP. Test card Lot 133 was also used to monitor platelet aggregation in the two ex vivo samples treated with heparin. The TAS prototype, fitted with the aluminum block for image capture, was used to monitor platelet aggregation. The video images were recorded. The start, stop and collapse time was recorded. For the purposes of this test, collapse time was defined as the time in minutes for the open ring structure initially formed by the PIOP after sample addition to change into a solid dot without a center opening. In the absence of collapse, the image of the test card was recorded for approximately 8 minutes.

The amount of light reflected from the surface of a test card was monitored in the ReoPro treated samples from 8 donors by measuring the DC voltage output from a TAS analyzer. This was performed by carefully removing the test card from the prototype TAS at the conclusion of the assay and covering the center of rotation with a ⅛^{th} inch square of white paper. The test card was then re-inserted into a TAS analyzer fitted with a standard sensor head to measure the light reflected from the surface of the card. This measurement was indicative of the total amount of vWf-coated PIOP transported from the outer edge of the reaction chamber into the middle of the chamber over the timeframe of the assay.

The results of the donor study are summarized in Table 2. This table is organized by donor. Age, sex and pertinent medications are indicated for each participant. The absolute value of the whole blood impedance measurement (IMP) using the Chrono-Log instrument are shown along with the percent inhibition of the whole blood impedance (INH %). If collapse was observed using the prototype TAS, the time was recorded in minutes and seconds. All test results using the prototype TAS were collected using card Lot 134. In those cases where reflectivity measurements were made on test cards, the DC voltage is shown.

TABLE 2

| | | | | ReoPro (ug/mL) | | | | | |
|---|---|---|---|---|---|---|---|---|---|
| # | Age | Sex | | 0 | 0.5 | 1 | 2 | 10 | Medications |
| 1 | 42 | Female | IMP | 16 | 14 | 15 | 12 | 0 | antiepileptics |
| | | | INH % | 0 | 13 | 6 | 25 | 100 | |
| | | | Collapse | 1:25 | 1:37 | 2:20 | None | None | |
| 2 | 53 | Male | IMP | 24 | 15 | 10 | 3 | 0 | naproxen, terazosin |
| | | | INH % | 0 | 38 | 58 | 88 | 100 | |
| | | | Collapse | 1:00 | 1:18 | None | None | None | |
| 3 | 48 | Male | IMP | 16 | 12 | 14 | 3 | 0 | metaproloc, isosorbide, |
| | | | INH % | 0 | 25 | 13 | 81 | 100 | atovvastatin, ASA |
| | | | Collapse | 1:04 | 1:10 | None | None | — | |
| 4 | 47 | Male | IMP | 22 | 18 | 16 | 9 | 0 | Ibuprofen |
| | | | INH % | 0 | 18 | 27 | 59 | 100 | |
| | | | Collapse | 1:02 | 0:57 | 1:08 | None | None | |
| 5 | 33 | Male | IMP | 19 | 16 | 15 | 10 | 0 | Ibuprofen |
| | | | INH % | 0 | 16 | 21 | 47 | 100 | |
| | | | Collapse | 0:59 | 0:59 | 0:36 | None | None | |
| 6 | 37 | Male | IMP | 18 | 12 | 12 | 2 | 0 | None |
| | | | INH % | 0 | 33 | 33 | 89 | 100 | |
| | | | Collapse | 0:58 | 1:08 | None | None | — | |
| 7 | 25 | Male | IMP | 18 | 14 | 12 | 2 | 0 | Ibuprofen |
| | | | INH % | 0 | 22 | 33 | 89 | 100 | |
| | | | Collapse | 1:40 | 1:06 | 1:23 | None | None | |
| 8 | 52 | Male | IMP | 16 | 17 | 17 | 4 | 0 | ASA |
| | | | INH % | 6 | 0 | 0 | 76 | 100 | |
| | | | Collapse | 0:50 | 0:45 | 1:12 | None | None | |
| 9 | 33 | Female | IMP | 17 | 12 | 15 | 6 | 0 | None |
| | | | INH % | 0 | 29 | 12 | 65 | 100 | |
| | | | Collapse | 1:00 | 0:51 | 1:00 | None | None | |
| 10 | 48 | Female | IMP | 20 | 14 | 15 | 6 | 0 | ASA |
| | | | INH% | 0 | 30 | 25 | 70 | 100 | |
| | | | Collapse | 1:39 | 0:50 | 1:45 | None | None | |
| 11 | 29 | Female | IMP | 19 | 13 | 11 | 2 | 0 | |
| | | | INH % | 0 | 32 | 42 | 89 | 100 | |
| | | | Collapse | 0:27 | 0:35 | 1:48 | None | None | |
| 12 | 34 | Female | IMP | 20 | 14 | 11 | 8 | 0 | None |
| | | | INH % | 0 | 30 | 45 | 60 | 100 | |
| | | | Collapse | 1:00 | 1:04 | 1:39 | None | None | |
| 13 | 47 | Female | IMP | 18 | 15 | 15 | 14 | 0 | None |
| | | | INH % | 0 | 17 | 17 | 22 | 100 | |
| | | | Collapse | 0:55 | 0:45 | 0:54 | None | None | |
| | | | DC Volts | 1.73 | 1.69 | 1.76 | 1.43 | 1.32 | |
| 14 | 44 | Female | IMP | 22 | 16 | 13 | 6 | 0 | ASA |
| | | | INH % | 0 | 27 | 41 | 73 | 100 | |
| | | | Collapse | 0:51 | 0:52 | None | None | — | |
| | | | DC Volts | 1.63 | 1.78 | 1.39 | 1.32 | — | |
| 15 | 25 | Male | IMP | 19 | 13 | 9 | 4 | 0 | None |
| | | | INH % | 0 | 32 | 53 | 79 | 100 | |
| | | | Collapse | 2:12 | 1:13 | None | None | — | |
| | | | DC Volts | 1.54 | 1.61 | 1.48 | 1.34 | — | |
| 16 | 53 | Female | IMP | 14 | 12 | 11 | 5 | 0 | None |
| | | | INH % | 0 | 14 | 21 | 64 | 100 | |
| | | | Collapse | 0:40 | 0:49 | 2:08 | None | None | |
| | | | DC Volts | 1.58 | 1.62 | 1.47 | 1.35 | 1.31 | |
| 17 | 35 | Female | IMP | 16 | 11 | 10 | 4 | 0 | None |
| | | | INH % | 0 | 31 | 38 | 75 | 100 | |
| | | | Collapse | 0:55 | 0:45 | None | None | — | |
| | | | DC Volts | 1.65 | 1.70 | 1.33 | 1.25 | — | |
| 18 | 39 | Female | IMP | 17 | 14 | 13 | 10 | 0 | acetaminophen |
| | | | INH % | 0 | 18 | 24 | 41 | 100 | |
| | | | Collapse | 0:48 | 0:51 | 1:23 | None | — | |
| | | | DC Volts | 1.75 | 1.68 | 1.46 | 1.22 | — | |
| 18 | 39 | Female | IMP | 20 | 13 | — | — | — | acetaminophen |
| | | 1U/mL | INH % | 0 | 35 | — | — | — | |
| | | Heparin | Collapse | 1:18 | 0:46 | 0:49 | None | — | |
| 19 | 46 | Male | IMP | 16 | 12 | 15 | 7 | 0 | None |
| | | | INH % | 0 | 25 | 6 | 56 | 100 | |
| | | | Collapse | 1:08 | 1:06 | 1:39 | None | — | |
| | | | DC Volts | 1.67 | 1.60 | 1.60 | 1.36 | — | |
| 19 | 46 | Male | IMP | — | — | — | — | — | None |
| | | 1U/mL | INH % | — | — | — | — | — | |
| | | Heparin | Collapse | 0:39 | 1:16 | 1:15 | None | — | |
| 20 | 44 | Male | IMP | 21 | 15 | 13 | 2 | 0 | None |
| | | | INH % | 0 | 29 | 38 | 90 | 100 | |
| | | | Collapse | 0:52 | 0:57 | 1:29 | None | — | |
| | | | DC Volts | 1.67 | 1.55 | 1.45 | 1.29 | — | |

As expected, platelet aggregation as measured by impedance was completely inhibited in all of the 20 ex vivo samples treated with 10 ug/mL ReoPro. At lower doses of ReoPro, however, the impedance response was quite variable. Inhibition of platelet impedance aggregation at 2 ug/mL of ReoPro varied from 22 to 90%, with 70% or more inhibition in 11 of the 20 patients. Using the TAS prototype, on the other hand, there was no collapse of the PIOP ring in any of the samples treated with 2 or 10 ug/mL ReoPro.

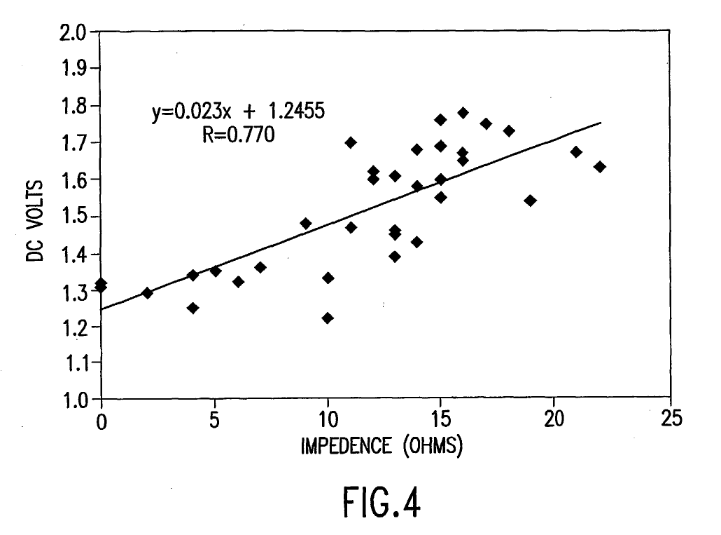

At the 1 ug/mL treatment level of ReoPro, the whole blood impedance aggregation varied from 0 to 58%. Collapse of PIOP was observed in 14 of the 20 samples treated with 1 ug/mL of ReoPro using the prototype TAS. The average impedance aggregation was significantly higher (39%, p=0.03) in the six samples for which no collapse was observed using the TAS prototype than for the 14 samples that did collapse (23%). Collapse of the PIOP ring was observed in all of the untreated samples as well as all of those treated with 0.5 ug/mL ReoPro. For those tests in which collapse was observed, the time ranged from as short as 0:27 to as long as 2:20. There was no apparent correlation between ReoPro treatment and collapse time. The DC voltage measurements recorded at the conclusion of the test on the TAS analyzer, however, did correlate to electrical impedance measurements made with the Chrono-Log instrument as shown in FIG. 4. This dramatically illustrates that as electrical impedance is reduced by the platelet antagonist ReoPro, less and less PIOP remains on the outside edges of the reaction area in the prototype TAS platelet function test.

Whole blood platelet aggregation, as measured by the Chrono-Log instrument or the prototype TAS, did not appear to be influenced by aspirin (ASA donors 3, 8, 10, & 14). Unfractionated heparin added at 1 U/mL did not adversely affect the platelet function assay performed on the prototype TAS using test card Lot 133 or 134 (Donors 18 & 19).

This application is based on U.S. Provisional application No. 60/202,638, filed with the U.S. Patent Office on May 9, 2000, the entire contents of which are hereby incorporated by reference.

Obviously, numerous modifications and variations of the present invention are possible in light of the above teachings. It is therefore to be understood that within the scope of the appended claims, the invention may be practiced otherwise than as specifically described herein.

1. A platelet function assay reagent, comprising:
a mixture of magnetic and non-magnetic particles, wherein said magnetic particles have bound to an outer surface thereof an amount of a first ligand having an affinity for direct interaction with GP-Ib receptors on blood platelets and wherein said non-magnetic particles have bound to an outer surface thereof an amount of a second ligand having an affinity for direct interaction with GP-Ib receptors on blood platelets, such that interaction of either of said first or second ligands with the GP-Ib platelet receptor will activate said blood platelets toward aggregation, wherein said first ligand and said second ligand can be the same or different.

2. The platelet function assay reagent of claim 1, wherein said first ligand is a member selected from the group consisting of von Willebrand factor, collagen, thrombin, and active fragments thereof.

3. The platelet function assay reagent of claim 2, wherein said first ligand is von Willebrand factor or an active fragment thereof.

4. The platelet function assay reagent of claim 1, wherein said second ligand is a member selected from the group consisting of von Willebrand factor, collagen, thrombin and active fragments thereof.

5. The platelet function assay reagent of claim 4, wherein said second ligand is von Willebrand factor or an active fragment thereof.

6. The platelet function assay reagent of claim 1, wherein said first ligand and said second ligand are the same.

7. The platelet function assay reagent of claim 6, wherein said first ligand and said second ligand are each von Willebrand factor or an active fragment thereof.

8. The platelet function assay reagent of claim 1, wherein said first ligand and said second ligand are different from one another.

9. The platelet function assay reagent of claim 8, wherein one of said first ligand or said second ligand is von Willebrand factor or an active fragment thereof.

10. A platelet function assay method, comprising:
contacting a whole blood or platelet rich plasma sample with a platelet function assay reagent in the presence of an oscillating or rotating magnetic field, said platelet function assay reagent comprising a mixture of magnetic and non-magnetic particles, wherein said magnetic particles have bound to an outer surface thereof an amount of a first ligand having an affinity for direct interaction with GP-Ib receptors on blood platelets and said non-magnetic particles have bound to an outer surface thereof an amount of a second ligand having an affinity for direct interaction with GP-Ib receptors on blood platelets, such that interaction of either of said first or second ligands with the GP-Ib platelet receptor will activate said blood platelets toward aggregation, wherein said first ligand and said second ligand can be the same or different;
monitoring movement of the magnetic particles in response to the oscillating or rotating magnetic field, to determine the presence or absence of platelet function, the level of platelet function, or both, in the whole blood or platelet rich plasma sample.

11. The method of claim 10, wherein said sample is whole blood.

12. The method of claim 10, wherein said sample is platelet rich plasma.

13. The method of claim 10, wherein said first ligand is a member selected from the group consisting of von Willebrand factor, collagen, thrombin, and active fragments thereof.

14. The method of claim 13, wherein said first ligand is von Willebrand factor or an active fragment thereof.

15. The method of claim 10, wherein said second ligand is a member selected from the group consisting of von Willebrand factor, collagen, thrombin and active fragments thereof.

16. The method of claim 15, wherein said second ligand is von Willebrand factor or an active fragment thereof.

17. The method of claim 10, wherein said first ligand and said second ligand are the same.

18. The method of claim 17, wherein said first ligand and said second ligand are each von Willebrand factor or an active fragment thereof.

19. The method of claim 10, wherein said first ligand and said second ligand are different from one another.

20. The method of claim 19, wherein one of said first ligand or said second ligand is von Willebrand factor or an active fragment thereof.

21. The method of claim 10, wherein said contacting occurs in the presence of a rotating magnetic field.

22. The method of claim 21, wherein said rotating magnetic field is rotating at a frequency of from 2000-3000 rpm.

23. A platelet function assay method, comprising:
contacting a whole blood or platelet rich plasma sample with a platelet function assay reagent in the presence of an oscillating magnetic field, said platelet function assay reagent comprising magnetic particles having bound to an outer surface thereof an amount of a ligand having an affinity for direct interaction with GP-Ib receptors on blood platelets, such that interaction of said ligand with the GP-Ib platelet receptor will activate said blood platelets toward aggregation;
monitoring movement of the magnetic particles in response to the oscillating magnetic field, to determine the level of platelet function in the whole blood or platelet rich plasma sample.

24. The method of claim 23, wherein said ligand is a member selected from the group consisting of von Willebrand factor, collagen, thrombin, and active fragments thereof.

25. The method of claim 24, wherein said ligand is von Willebrand factor or an active fragment thereof.
